(19) United States
(12) Patent Application Publication
(10) Pub. No.: US 2023/0375328 A1
(43) Pub. Date: Nov. 23, 2023

(54) ARTICLES AND METHODS FOR OSTEOMETRIC MEASUREMENT

(71) Applicant: Mississippi State University, Starkville, MS (US)

(72) Inventor: Eric Anderson, Starkville, MS (US)

(21) Appl. No.: 18/198,817

(22) Filed: May 17, 2023

Related U.S. Application Data
(60) Provisional application No. 63/343,022, filed on May 17, 2022.

(51) Int. Cl. G01B 11/02; G01S 7/481; G01S 17/08

(52) U.S. Cl. CPC G01B 11/02; G01S 7/4814; G01S 17/08

(57) ABSTRACT

An osteometric measuring device that comprises a first panel; a second panel movably positioned with respect to the first panel; and a light emitting device arranged and disposed in the first panel to measure distance by time-of-flight; wherein the light is emitted from the first panel, reflected off of the second panel, and received by the first and measured to calculate the distance from the first panel to the second.

### TECHNICAL FIELD

The present invention relates to articles and methods for osteometric measurement. In particular, the presently-disclosed subject matter relates to a portable osteometric measurement devices for measuring artifact and bone length, as well as methods of measurement using the same.

### BACKGROUND AND SUMMARY

Anthropometry centers on methods for measuring the human body and has been used since the early 18th century to understand more about our species (Jamison and Zegura 1974; Marks 2012, 2017). Anthropometric data collected from human skeletal material, or osteometric data, give biological anthropologists, human osteologists, bioarchaeologists, and forensic anthropologists the means to estimate sex and maximum living stature as well as quantifying differences in growth and development (e.g., long bone metrics) (e.g., Adams and Byrd 2002; DiGangi and Moore 2012; Moore 2012; Moore and Ross 2012). They can also use this information to compare their findings with metrics from other osteological analyses, which makes the reliability (i.e., when an object is measured on two different occasions by at least two different observers, with little random error) and validity (i.e., degree to which a measurement consistently accomplishes its intended purpose) of these measurements especially important. This type of information must be collected methodically due to human error (both inter- and intra-observer error), instrumentation issues, and the observer's experience.

The osteometric board is the one of the primary devices used for recording osteometric data from human skeletal material, such as long bone length. Notably though, there have been few changes from the osteometric board's original 19th century design, which can be bulky and inconvenient to transport. One example of these changes is the Abawerk osteometric board, which has two upright panels along the length and width of the board with metric ruled paper placed on the base. The Abawerk osteometric board is used to determine the length of skeletal material placed on the board (Geise 1986). This board design makes it easier to generate measurements and increases their precision because the ruled paper allows measurements to be taken from multiple angles (Geise 1986). Commercially manufactured osteometric boards, such as those by Carolina Supply Company and Paleo-Tech, have made strides in developing more portable models by making them collapsible, but they are expensive. To counter this cost, some researchers have made their own osteometric boards. Naples and colleagues (2010) for example, created an inexpensive osteometric board for the classroom. Their goal was to facilitate students' usage of osteometric boards, offering more opportunities to learn forensic methods. This osteometric board consisted of rulers, tape, glue, and cardboard (Naples et al. 2010). While this option is highly affordable, it is not particularly durable.

In addition to the design limitations, inter- and intra-observer variation must be considered by researchers when examining osteometric data (Adams and Byrd 2002). The inter-observer error results from inconsistent measurements between different observers, while intra-observer error results from inconsistent measurements taken by a single observer (Adams and Byrd 2002). Generally, inter-observer error is greater than intra-observer error, and fewer errors will occur with the measurement of maximum length and breadth measurements of skeletal material (Langley et al. 2018). Acceptable error rates for anthropometry are <1.5% for intra-observer error and <2% for inter-observer error (Perini et al. 2005). Even those postcranial measurements classified as difficult for most observers to generate, like those of the tibia, should have less than a 3% error rate (Adams and Byrd 2002).

Failure to understand what factors can impact an osteometric board, like humidity, will lead to errors with the measurements due to the instrument or observer (Langley et al. 2018). Some factors are external to the observer, while some are specific to the observer. One example of an external factor is the effects of humidity on the Abawerk osteometric board (Geise 1986). Giese found that this type of board's measurement results change depending on the amount of humidity present in the local environment during data recording. Specifically, Giese found that elevated relative humidity, of 60% and higher, made the grid paper expand and changed the measurement's outcome between 0.5 and 1 mm. Overall, as ambient humidity increased, so did the length of the measurements (Geise 1986). Conditions specific to the observer include the amount of experience the observer has using an osteometric board, which may generate poor reliability within the collected data. For example, Adams and Byrd (2002) conducted a study with 68 participants during the 52nd American Academy of Forensic Sciences meeting (AAFS). These participants, who had various amounts of experience with recording postcranial measurements, used both digital calipers and an osteometric board to record 22 postcranial measurements. They found that those with less experience produced measurements with increased inter-observer error, especially those with less than five years of experience. The common errors they identified in the study included transposing numbers, decimal place errors, not zeroing out the calipers, incorrect measurement transcription, and not understanding how to take measurements following established standards (e.g., Buikstra and Ubelaker 1994). In sum, Adams and Byrd (2002) showed that experience with the equipment would increase proficiency to a certain point. However, complacency can also occur if the observer is overconfident in their skills and fails to conduct the measurements carefully (Adams and Byrd 2002).

In osteometric analysis, reliability and replicability are fundamental (Adams and Byrd 2002; Langley et al. 2018). One way to accomplish this is by restricting analyses to osteometric measures that do not require extensive experience, like maximum length measurements (Adams and Byrd 2002). These types of measurements have lower error possibilities and can help decrease inter- and intra-observer errors (Adams and Byrd 2002). However, this can greatly limit the scope of analyses incorporating osteometric data. Also important is recognizing extrinsic or environmental factors such as humidity that may impact error and accuracy (Albrecht 1983; Langley et al. 2018; Geise 1986).

Accordingly, there remains a need in the art for portable, reliable, and affordable osteometric measurement devices.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIG. 1 shows an image illustrating a portable osteometric measurement device of the present invention.

FIGS. 2A-D show images of osteometric measurements in connection with the measurement table in the Specification. (A) Measuring the maximum length of the humerus. (B) Measuring the maximum length of the ulna. (C) Measuring the maximum length of the femur. (D) Measuring the maximum length of the tibia.

FIG. 3 shows an image illustrating a portable osteometric measurement device.

FIG. 4 shows an image of a Paleo-Tech Lightweight Field Osteometric Board.

FIGS. 5A-C show images illustrating components of an osteometric measurement device. (A) Laser panel. (B) Sliding panel. (C) Calibrating block.

FIGS. 6A-D show images illustrating components of an osteometric measurement device. (A) Perspective view of laser panel. (B) Rear view of laser panel. (C) Side view of laser panel. (D) Side view of sliding panel.

FIG. 7 shows images illustrating assembly of an osteometric measurement device laser panel.

FIG. 8 shows images illustrating assembly of an osteometric measurement device sliding panel stabilizer.

FIG. 9 shows images illustrating mounting of an osteometric measurement device.

FIG. 10 shows an image illustrating control buttons on an osteometric measurement device.

FIG. 11 shows images illustrating measurement with an osteometric measurement device.

FIG. 12(A)-(D) are mean and standard deviation graphs.

FIG. 13 is a view of the first and second panel attached together for transportation or storage.

FIG. 14 is a image showing the first and second panels measuring a bone.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

Although any methods, devices, and materials similar or equivalent to those described herein can be used in the practice or testing of the presently-disclosed subject matter, representative methods, devices, and materials are described herein.

Following long-standing patent law convention, the terms “a”, “an”, and “the” refer to “one or more” when used in this application, including the claims, unless the context clearly dictates otherwise. Thus, for example, reference to “a polypeptide” includes one or more of such polypeptides, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

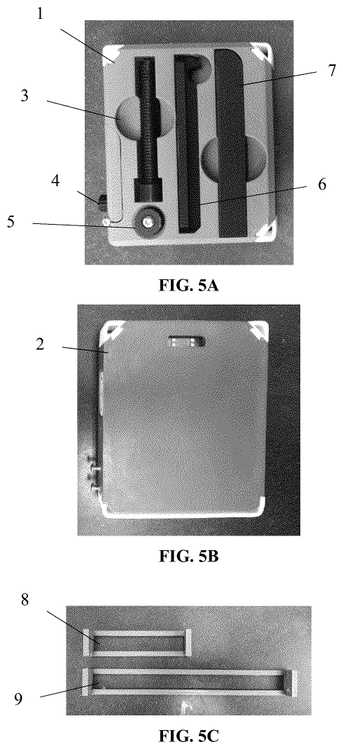

Provided herein are osteometric measurement devices. In some embodiments, the osteometric measurement device includes a first panel (See FIG. 5B, for example), a second panel (see FIG. 5A, for example) movably positioned with respect to the first panel, and a light emitting device. The light emitting device includes a transmitter configured to emit a light and a receiver configured to receive the emitted light. Using time-of-flight (ToF) technology, a distance is measured by determining the time it takes for photons to travel from the transmitter to the receiver. In some embodiments, the transmitter and the receiver are positioned in the first panel the second panel is configured to reflect a light produced by the light emitting device of the first panel. In such embodiments, the emitted light is reflected off of the second panel and back to the first panel where it is received by the receiver. Alternatively, in some embodiments, the first panel includes the transmitter and the second panel includes the receiver for directly receiving the emitted light with first reflecting it.

Suitable light emitting devices include, but are not limited to, an infrared laser, a red laser, any other suitable laser or laser sensor, or any other suitable light emitting device for use in connection with time of flight (ToF) measurement. For example, in one embodiment, the laser includes a red laser (e.g., VL53L0X laser sensor available from STMicroelectronics) and the second panel includes any solid surface for the laser to reflect off of. In another example, the laser includes an infrared (IR) laser and the second panel includes an IR reflective surface. In some embodiments, the second panel includes a white filament with a reflectivity of at least 73%. In some embodiments, the reflectivity of at least 73% provides an error rate of less than 2%. In one embodiment, the second panel includes a reflectivity of at least 88%. The panels may be formed by any suitable method depending upon the material used, such as, but not limited to, casting, molding, three-dimensional (3D) printing, or any other suitable method.

The first panel and the second panel are configured for placement on any flat surface. In some embodiments, the first panel and the second panel are movably coupled to each other. For example, in one embodiment, the first panel includes a sliding feature extending therefrom and the second panel is arranged and disposed to engage the sliding feature extending from the first panel. The sliding feature includes and suitable element on which the second panel can slide, such as, but not limited to, a bar, two or more parallel bars, a track, or any other element that is capable of extending along the flat surface and engaging with the second panel. In addition to serving as a guide for the movement of the second panel with respect to the first panel, in some embodiments, the sliding feature also couples the second panel to the first panel.

Additionally or alternatively, in some embodiments, the first panel and/or the second panel are removably fixed to the flat surface. In one embodiment, for example, the first panel is secured to a clamp, which may be secured to the flat surface. In another embodiment, the second panel includes a stabilizer arranged and disposed to contact the flat surface and stabilize the second panel. Suitable stabilizers include, but are not limited to, flaps, extensions, or overhanging portions that adjustably contact the flat surface to stabilize the second panel. For example, in some embodiments, the second panel is secured to the sliding feature extending from the first panel, and the stabilizer includes an overhang with an adjustable screw. In such embodiments, once the second panel is coupled to the sliding feature, the screw in the overhang is adjusted to contact the side of the flat surface such that the sliding feature and the overhand together stabilize the second panel with respect to the flat surface and the first panel.

In some embodiments, the osteometric measurement device also includes a display screen (e.g., LCD), a power switch, a control panel, and/or a charging socket. For example, in some embodiments, the first panel includes a display screen configured to display operating conditions and/or measurements and a control panel coupled to the display screen for programming/operating the device. Additionally or alternatively, the osteometric measurement device may be battery powered and/or plugged into an electrical socket. For example, in one embodiment, the device is battery powered and includes a charging socket for plugging the device in and charging the battery. In another embodiment, the charging socket may be used to directly power use of the device, such as, for example, when the battery is low on charge. A power switch is also included to turn the device on and off.

Although described herein primarily with respect to the display screen, power switch, control panel, and/or charging socket being on the first panel, the disclosure is not so limited and may include any one or more of these features on the second panel or a separate panel. For example, in some embodiments, the sliding feature may electrically couple the first panel and the second panel such that any of the electrically powered features may be included in either or both panels. Furthermore, in some embodiments, the device may include a motor arranged and disposed to move the second panel with respect to the first panel. For example, in one embodiment, the sliding feature includes a motorized track for moving the second panel. In another embodiment, the second panel includes a motor for moving the panel along the sliding feature.

Once the first panel and the second panel are positioned relative to and/or coupled to each other, an article to be measured is positioned therebetween. The second panel is then moved relative to the first panel until both the first panel and the second panel are contacting opposite portions of the article to be measured. The light emitting device is then activated and time-of-flight of the emitted light is used to measure the distance between the panels, which also provides a measurement of the article positioned between the panels. The article includes any suitable article to be measured, such as, but not limited to, bones, archeological artifacts, or any other suitable article to be measured.

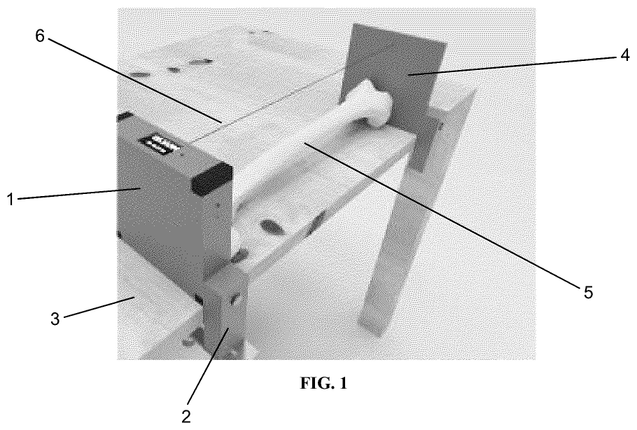

Turning to the figures, FIG. 1 shows the first panel (1) clamped (2) to a table (3). The second panel 4 is also clamped to the table. A bone 5 is snugly positioned between the first and second panel. A laser beam 6 is emitted from the first panel, reflects off the second panel back to the first, and a distance is measured that is equal to the length of the bone.

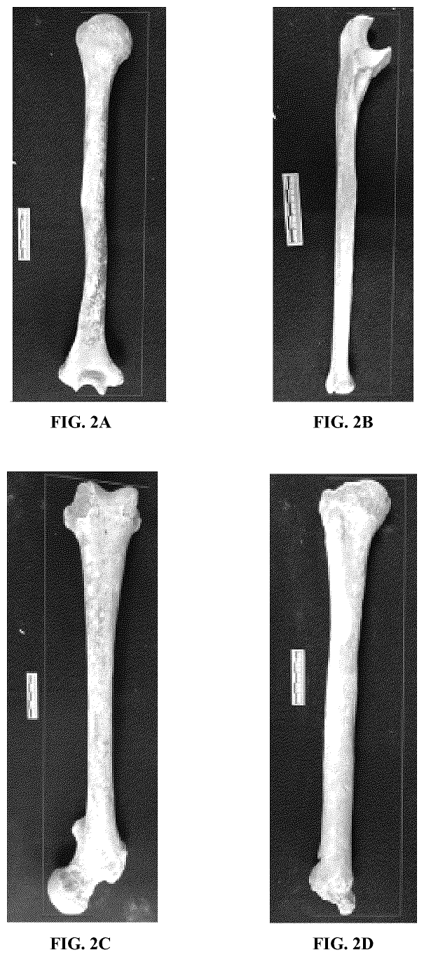

FIG. 2 shows various bones measured by the device of the present invention.

FIG. 5 shows a panel that includes a housing that incorporates the clamping mechanism. In this case, the clamp screw (4), clamp sliding bar screw (5) clamp sliding bar (5), and the stabilizer (7) all fit within compartments of the panel. For ease of transportation, the panels can be attached together.

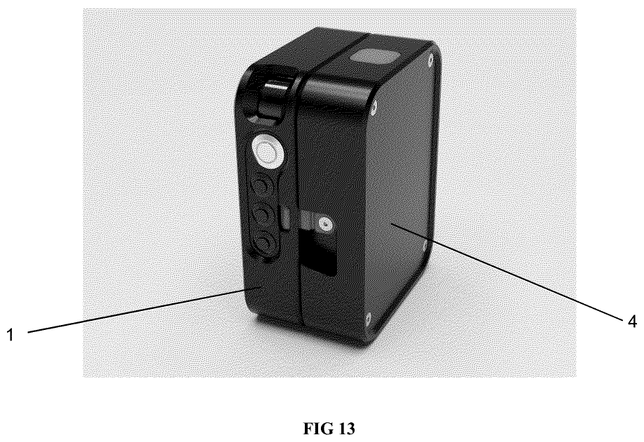

FIG. 13 shows the first panel (1) attached to the second panel (4) for storage and transportation. The reflection target (7) for a light beam, such as a laser beam, is shown.

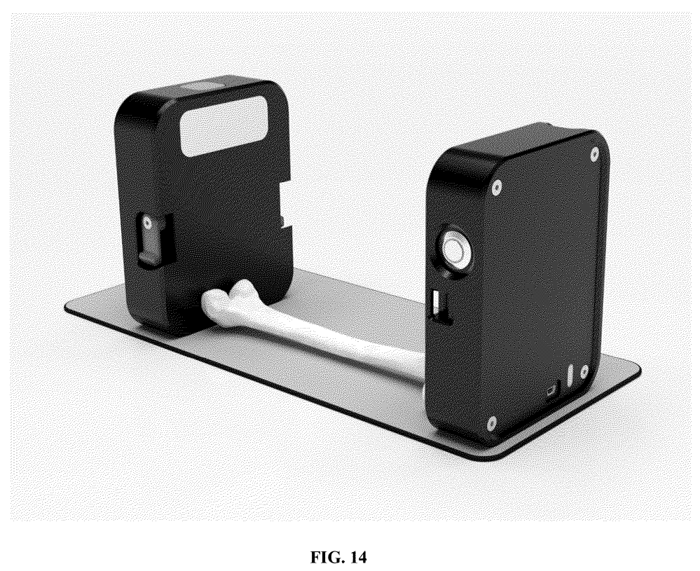

FIG. 14 shows the first and second (1), (4), engaging a flexible mat (6) to assist with stabilization and alignment. The length of a bone is being measured.

Without being bound by theory or mechanism, it is believed that the devices disclosed herein provide increased portability, accuracy, precision, and ease of measurement as compared to existing devices. More specifically, as the first panel and the second panel may be positioned on and/or attached to any flat surface, there is no need for a full length board as with the existing osteometric measurement boards, making the device smaller and more portable than existing devices. This small size (e.g., 152.4×152.4×25.4 mm) makes the device easier to transport for researchers traveling to conduct fieldwork and conducting data collection in field settings. The smaller size also makes the device easier to store and takes up less space than other conventional osteometric boards. Additionally, the open concept provides the ability to measure various artifacts and nonhuman remains at smaller sizes which are not possible with conventional osteometric boards (e.g., from 30 mm to 2 m). Furthermore, the devices disclosed herein use time-of-flight measurement that does not require visual measurement by the user, which reduces or eliminates this aspect of user error/inconsistency seen with the existing osteometric measurement boards.

Also provided herein are methods of measuring an artifact using the device according to one or more of the embodiments disclosed herein. In some embodiments, the method includes positioning the first panel and the second panel, positioning the artifact to be measured between the first panel and the second panel, moving the second panel with respect to the first panel such that each panel touches an opposite side of the artifact, emitting a light from the light emitting device, and measuring the distance between the first panel and the second panel using direct ToF based upon the emitted light. Since the first panel and the second panel are contacting opposite sides of the artifact, the distance between the first panel and the second panel is equal to the measurement of the artifact. In some embodiments, positioning the first panel includes connecting the first panel to a clamp stabilizer, such as, for example, with a bolt. The clamp stabilizer is then secured to a flat surface, such as a table, positioning the first panel with respect to the flat surface (e.g., at a 90° angle). In some embodiments, the second panel is then secured to the sliding feature of the first panel, moveably coupling the second panel to the first panel. Next, the stabilizer of the second panel is adjusted to stabilize the second panel with respect to the flat surface. An object for measurement (e.g., skeletal material or other artifact) is positioned against the first panel and the second panel is moved to sandwich the object between the two panels. The light emitting device is then activated and the measurement is displayed on the display screen.

In another embodiment, the portable osteometric device of the present invention may have Bluetooth capabilities to allow collected data to transfer to an application program (APP) automatically. The APP can be customizable to allow our customers to tailor their data collection to meet their different research goals. This APP will store all the collected data online and offline until the researcher uploads it into their database. This automatic transfer of data would minimize transcription errors and decrease data entry time, which would allow for increased time to analyze the data for research.

The presently-disclosed subject matter is further illustrated by the following specific but non-limiting examples. The following examples may include compilations of data that are representative of data gathered at various times during the course of development and experimentation related to the presently-disclosed subject matter.

### EXAMPLES

### Example 1—Osteometric Laser—First Study

This Example describes testing of the reliability and replicability of the osteometric measurement device disclosed herein against the Paleo-Tech Lightweight Field Osteometric board by providing measurements of intra-observer and inter-observer error. A preliminary assessment aimed at determining the applicability of a direct ToF laser measuring device, was conducted in 2021 using a laser for the collection of osteometric data, specifically long bone length (Anderson and Osterholtz 2021). This study used a Bosch Blaze GLM 50 C laser measure (Bosch laser) to determine if bone length measurements taken using this device were comparable to those generated with a Paleo-Tech Lightweight Field Osteometric board. Twenty volunteers from within the Department of Anthropology and Middle Eastern Cultures who had both different levels of educational attainment (i.e., BA degree in progress, MA degree in progress, PhD attained) and varying levels of experience in collecting osteometric data were incorporated into this study. These volunteers measured cast replicas of a femur and a radius using both the Bosch laser and a PaleoTech osteometric board. A Pearson R test was used to compare the relationship between osteometric data collected using the Bosch laser and using the PaleoTech osteometric board by the volunteers. Both devices showed a small correlation with the measurements taken of the radius (r=0.597, n=20, p=0.005) and the femur (r=0.988, n=20, p=0.04). Only a small correlation was found between the measurements taken from the Bosch laser and the PaleoTech osteometric board. This small correlation is due to interobserver error associated with different level of observed experience, potentially exacerbated by the fact that observers were not given any instruction in how to take the osteometric measurements Also, by not being anchored to a table or board, the Bosch laser had some instability (much like Broca's original device), which likely caused some variation within the measurements when compared to the Paleotech osteometric board. The results showed that the present invention is comparable to the osteometric board.

### Osteometric Laser—Second Study

Methods

3D Modeling and Printing Methods

3D printing technology and supporting hardware was used to create the PODv1 housing unit for the laser. This production method was the least expensive method and the easiest method for creating the housing for the PODv1 laser. The PODv1 housing was designed in Autodesk Fusion 360 CAD software. This CAD software was chosen because it is a user-friendly operating and is open-source. Once the 3D model was rendered in CAD, the PODv1 was placed into the PrusaSlicer slicing software program to be transitioned into a series of layers and a format that the 3D printer can print. This slicing software was used because it was compatible with a 3D printer that was accessible. The Creality Ender-3 V2 FDM 3D Printer with ABS (Acrylonitrile Butadiene Styrene) filament printed the PODv1 housing. ABS filament was chosen because it is the least expensive material suitable for creating the PODv1 housing.

Baseline

Volunteers participating in this research were proctored and created the baseline measurement for each of the four bones elements, specified above. The four bone elements were drawn from the ÐurÐevac-Sošice Comingled Collection at MSU. This collection was chosen due to accessibility and proximity to the volunteers' location, permission of the descent community for inclusion of the remains within research contexts, and the completeness of the bone elements available. This baseline was accomplished using the traditional Paleo-Tech Lightweight Field Osteometric Board, and measuring each of the four bone elements three different times. Then the results were averaged to create a baseline measurement for each bone. These baseline measurements were used as the true measurement. Those measurements were then compared to the measurements made by the volunteers who used the PODv1 and the PaleoTech osteometric board in order to test interobserver error and validity.

Volunteers

Volunteers at Mississippi State University (MSU) collected osteometric data. The volunteers at MSU were required to have completed an undergraduate or graduate-level osteology course, whether at MSU or a previous degree-granting institution, or possess equivalent experience prior to data collection. In this project, equivalent experience is any training or work that focused on human remains (e.g., lab work and field school). All volunteers were given written instructions and watched a six-minute video, made by the author, on how to operate both measuring devices. Additionally, each volunteer was given written and pictorial instructions on how to conduct the measurements for each of the four bone elements. These instructions directly followed the procedures described in the “Data Collection Procedures For Forensic Skeletal Material 2.0” (Langley et al. 2016).

TABLE 1
The number of measurements that one volunteer will conduct.

| | Humerus | Ulna | Femur | Tibia | |
|---|---|---|---|---|---|
| P.O.D.v1 | 3 | 3 | 3 | 3 | |
| PaleoTech | 3 | 3 | 3 | 3 | |
| Total | 6 | 6 | 6 | 6 | 24 per person |

The volunteers recorded their bone length measurements using an Excel spreadsheet. In order to keep this project unbiased and to maintain anonymity each volunteer recorded their measurements, number of years of experience with an osteometric board, and their discipline of study (e.g., anthropology or forensics). No other personal information was recorded.

Measurements

The table below gives a description on how the humerus, ulna, femur, and tibia were measured.

| Bone | Measurement | Description | Source |
|---|---|---|---|
| Humerus | Maximum Length of | “Place the humerus on the osteometric | Measurement #45 |
| (FIG. 2A) | the Humerus | board so that its long axis parallels the | (N. Langley et al. |
| | “The distance from | instrument. Place the head of the | 2016, 74) drawing |
| | the most superior | humerus against the vertical end | on Hrdli\check{c}ka |
| | point on the head of | board and press the movable upright | (1920: 126) |
| | the humerus to the | against the trochlea. Move the bone | |
| | most inferior point on | up, down and sideways to determine | |
| | the trochlea.” | the maximum distance.” | |
| Ulna | Maximum Length of | “Place the proximal end of the ulna | Measurement #54 |
| (FIG. 2B) | the Ulna | against the vertical end board. Press | (N. Langley et al. |
| | “The distance between | the movable upright against the distal | 2016, 75-76) |
| | the most proximal | end while moving the bone up, down | drawing on Bräuer |
| | point on the olecranon | and sideways to obtain the maximum | and Hrdli\check{c}ka (1988 |
| | and the most distal | length.” | 204, #1; 1920: 127) |
| | point on the styloid | | |
| | process.” | | |
| Femur | Bicondylar Length of | “Place the femur on the osteometric | Measurement #76 |
| (FIG. 2C) | the Femur | board so that the bone is resting on its | (N. Langley et al. |
| | “The distance from | posterior surface. Press both distal | 2016, 78) drawing |
| | the most proximal | condyles against the vertical end | on Bräuer and |
| | point on the head of | board while applying the movable | Hrdli\check{c}ka |
| | the femur to a plane | upright to the head of the femur.” | (1988: 216, #2; |
| | drawn between the | | 1920: 128) |
| | inferior surfaces of the | | |
| | distal condyles.” | | |
| Tibia | Length of the Tibia | “Place the tibia on the osteometric | Measurement #86 |
| (FIG. 2D) | “The distance from | board resting on its posterior surface | (N. Langley et al. |
| | the superior articular | with the longitudinal axis of the bone | 2016, 81) drawing |
| | surface of the lateral | parallel to the board (Hrdli\check{c}ka 1920). | on Bräuer |
| | condyle of the tibia to | If using an osteometric board without | (1988: 220, #1) |
| | the tip of the medial | a hole, place the tibia on the | |
| | malleolus.” | osteometric board so that it the long | |
| | | axis is parallel to the board. The | |
| | | measurement is taken from the lateral | |
| | | condyle to the tip of the medial | |
| | | malleolus.” | |

Equipment

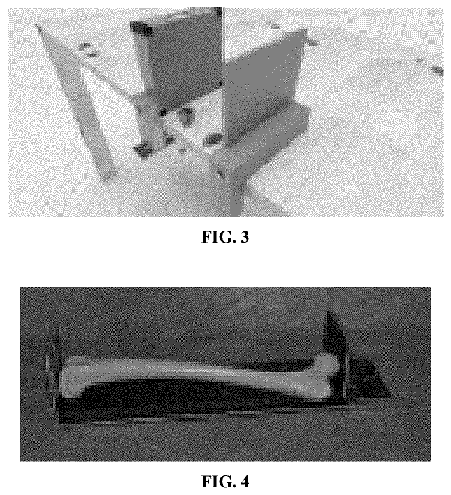

The table below lists and describes all the equipment that will be used.

| Equipment | Description | Technical Specifications |
|---|---|---|
| Portable Osteometric Device | This model consists of two main components | Dimensions - 152.4 × |
| Version 2 (P.O.D.v1) | that are the Laser Panel and Sliding Panel. | 152.4 × 25.4 mm |
| (FIG. 3) | The Laser Panel attaches to a table using a | Range - 30 mm-2 m |
| | clamp and the Sliding used to sandwich the | Power - Rechargeable |
| | bone that is wanted to be measured. The | batteries (120 hrs.) |
| | P.O.D. uses an internal laser sensor that uses | |
| | time-of-flight technology to measure the | |
| | distance between the two panels. It has two | |
| | different measuring options that are displayed | |
| | on an LCD screen: Live Measure and | |
| | Precision Mode. | |
| | The Live Measure displays a constant | |
| | maximum length and midpoint of the | |
| | distance. Precision Mode takes the average of | |
| | 50 measurements and displays it on the LCD | |
| | screen. | |
| Paleo-Tech Lightweight | This Osteometric board is a portable version | Dimensions - (Assembled) - |
| Field Osteometric Board | of the original design that folds up and is | 600 × 165.1 × 12.7 mm |
| (FIG. 4) | meant to be easier to transport. It uses a | (Folded) - 304.8 × |
| | guided Sliding Panel to manual record | 165.1 × 38.1 mm |
| | measurements. | Range - 1 mm-600 mm |
| | | Power - None |

The Portable Osteometric Device version 1 (POD) is a new measuring device that uses laser sensors with time-of-flight technology to measure distance. This device can be used to measure any object to determine its length. The POD has two modes of measuring a live and Precision mode measuring.

POD Specifications

Dimensions
Laser Panel—115×97×31 mm
Sliding Panel—115×97×24 mm

Range—00 mm-2 m

Power—Rechargeable battery (120 hrs.)

### PARTS AND COMPONENTS

### Parts

1. Laser Panel (FIG. 5A)
2. Sliding Panel (FIG. 5B)
3. Clamp Screw (FIG. 5A)
4. Stabilizing Screw (FIG. 5A)
5. Clamp Sliding Bar Screw (FIG. 5A)
6. Clamp Sliding Bar (FIG. 5A)
7. Stabilizer (FIG. 5A)
8. 100 mm Calibrating Block (FIG. 5C)
9. 200 mm Calibrating Block (FIG. 5C)

### Components

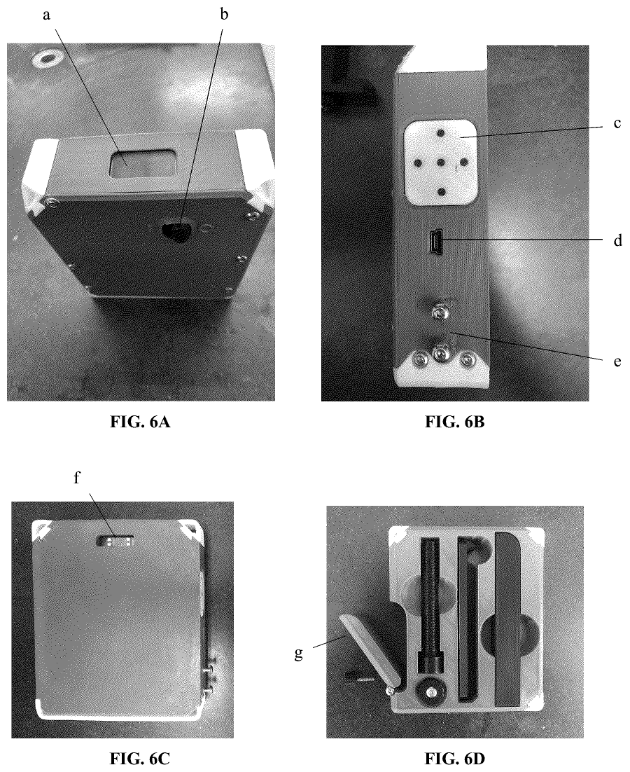

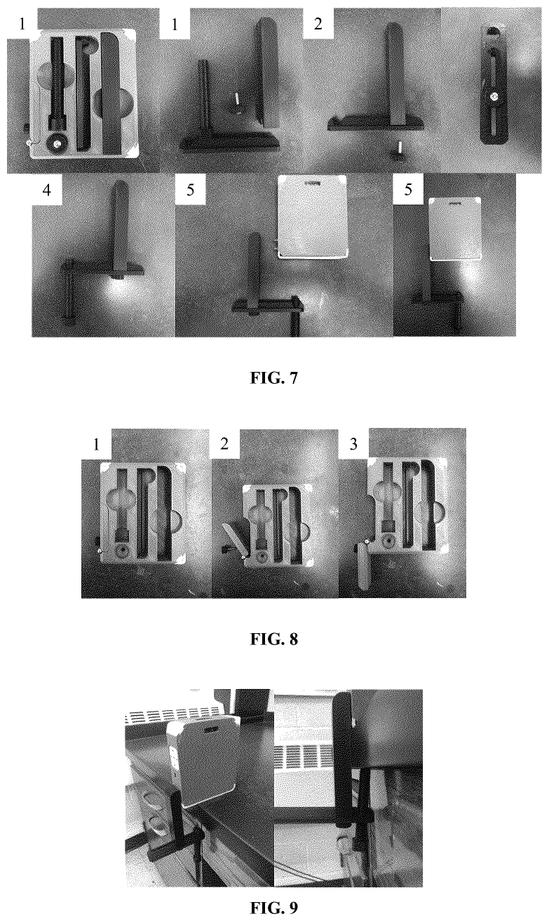

Laser Panel (FIGS. 6A-C)
A) Screen (FIG. 6A)
B) Power Switch (FIG. 6A)
C) Control Panel (FIG. 6B)
D) Charging Socket (FIG. 6B)
E) Stabilizer Hanger (FIG. 6B)
F) Laser Sensors (FIG. 6C)
Sliding Panel (FIG. 6D)
G) Stabilizer (FIG. 6D)
Assembly (FIG. 7)

Laser Panel Clamp
1. Remove all parts that are housed within the Sliding Panel.
Clamp Screw, Clamp Sliding Bar Screw, Clamp Sliding Bar, and Stabilizer
2. Place the Clamp Sliding Bar raised portion against the open portion of the Stabilizer.
3. Using the Clamp Sliding Bar Screw, fascine the Clamp Sliding Bar to the Stabilizer.
4. Screw the Clamp Screw into the hole found on the Clamp Sliding Bar Screw, so that knob portion is on the same side as the Clamp Sliding Bar Screw knob.
5. After the clamp has been assembled attach the clamp to the Laser Panel by aligning the two holes on the Stabilizer and the Stabilizer Hanger. Once these are aligned push them together and pulldown so that it secures the clamp to the Laser Panel.

Sliding Panel Stabilizer (FIG. 8)
1. Remove the Stabilizing Screw from the side of the Sliding Panel.
2. Unfold the Stabilizer.
3. Screw the Stabilizing Screw into the hole so that it secures the Stabilizer

Operating

Mounting (FIG. 9)
1. Place the assembled POD Laser Panel with clamp against a table so that the clamp is firmly against the side of the table.
2. Using the Clamp Sliding Bar, adjust the clamp to fit the table and tighten the Clamp Sliding Bar Screw to secure the adjusting bar.
3. After adjusting the clamp to the table that will be used tighten the Clamp Screw to secure the Laser Panel to the table.

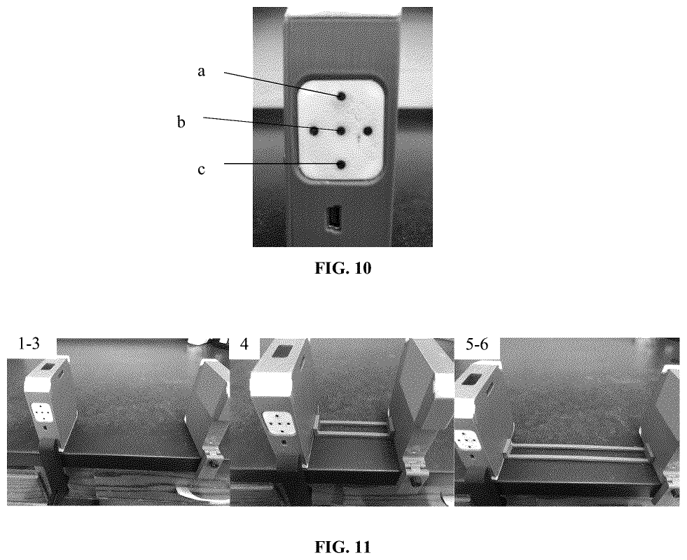

Buttons (FIG. 10)
A. Up/Measurement
B. Menu/Select
C. Down

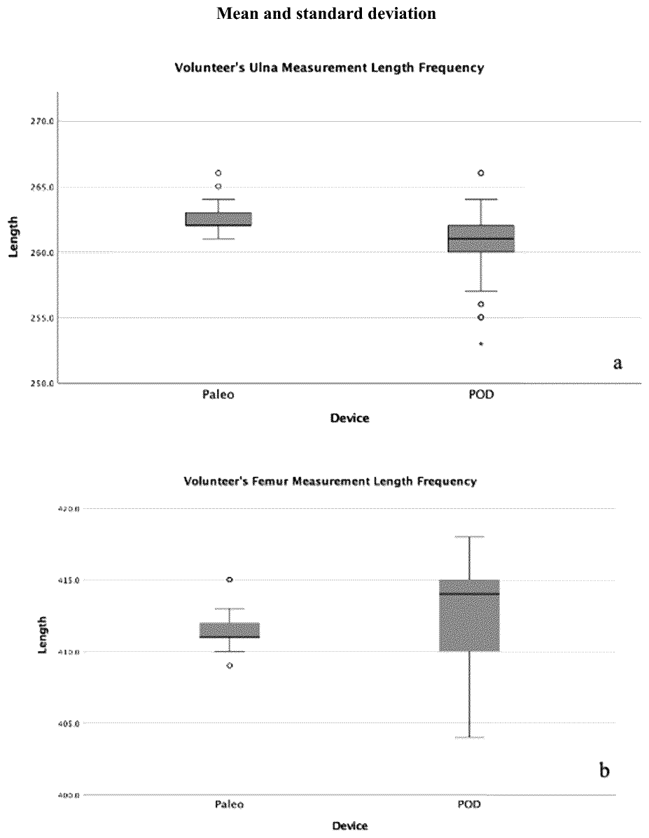

Measuring (FIG. 11)

Live Measurement
1. Attach the fully assembled POD against a table.
2. Turn on the POD and press the menu button.
3. Place the object that is to be measured against the POD Laser Panel.
4. Using the Sliding Panel sandwich the object between the two panels.
5. The live measurement will be then displayed on the LCD screen located on the Laser Panel

Precision Mode
1. Attach the fully assembled POD against a table.
2. Turn on the POD and press the menu button.
3. Place the object that is to be measured against the POD Laser Panel.
4. Using the Sliding Panel sandwich the object between the two panels.
5. Hold the Measurement button until the POD LCD screen begins counting down from 3. Once the countdown is complete the measurement will be completed and then displayed on the LCD screen located on the Laser Panel.

History
1. Turn on the POD and press the menu button.
2. Select the History program on the menu.
3. This will then show the last 5 Precision Mode measurements taken by the POD.

Calibrating
4. Attach the fully assembled POD against a table.
5. Turn on the POD and press the menu button.
6. Select the Calibration Mode
7. After starting the Calibration, you will be prompted to place the 100 mm Calibrating Block between the Laser Panel and Sliding Panel. Once the 100 mm Calibrating Block is even and snug between the two panels press the Select button to begin calibration.
8. Once the 100 mm calibration is complete, the POD will ask you to place the 200 mm Calibrating Block between the two panels. After the 200 mm Calibrating Block is even and snug between the two panels press the Select button to finish calibration.
9. After the POD has finished calibration it will tell you that it is complete.

Results

Volunteers took three different measurements of each bone element with both devices, which summed to 552 measurements. These measurements gave each bone element 138 independent measurements with 69 on each device.

Results for Between Devices

A Wilcoxon rank sum test with continuity correction was used to compare the volunteers' length measurements of the four bone elements taken using the PODv1 and the PaleoTech osteometric board (Wilcoxon 1945). The Wilcoxon rank sum test with continuity is used for these data because the variances of the two independent samples are not equal and they do not have a normal distribution. The length measurements of the tibia (w=2367, p-value=0.8082) and humerus (w=2629.5, p-value=0.2824) taken from both devices showed no statistical significance between them. Thus any difference in the present invention in measuring a tibia and a humerus was not statistically significant in the length measurements that they generate when compared to the Paleo-Tech Lightweight Field Osteometric Board. There was a statistically greater difference between the two devices' length measurements of the femur (w=1616.5, p-value=0.001025) and ulna (w=3550.5, p-value=0.0000003017).

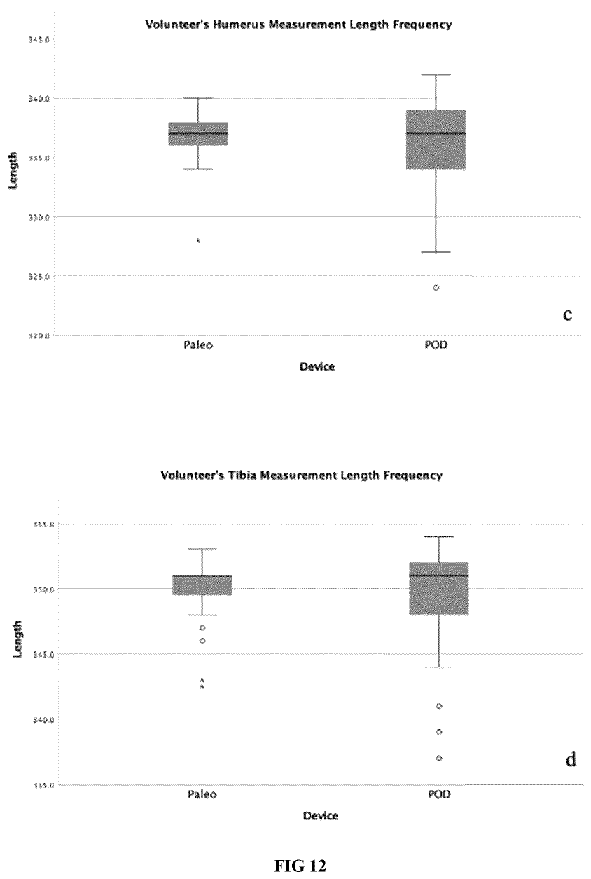

The mean and standard deviation rates were calculated for each of the four bones using both devices (See the table below and FIGS. 12(A) and 12(B)). Neither device means showed a larger or smaller mean length measurement trend. The standard deviation rates did show a trend, with the PODv1 having a larger value than the PaleoTech osteometric board. An exception from this trend was the standard deviation rate for the tibia made by the volunteers using the Paleotech (sd=16.23).

Mean and Standard Deviation:

| | |
|---|---|
| PODv1 | PaleoTech |

| Bone | Mean | sd | Mean | sd |
|---|---|---|---|---|
| Ulna | 260.68 | 2.422 | 262.348* | .9483* |
| Femur | 412.55 | 3.583 | 411.45 | 1.235 |
| Tibia | 349.51 | 3.433 | 350.216* | 1.895* |
| Humerus | 336.23 | 3.158 | 337.09 | 1.738 |

*One outlier removed from the ulna and two from the tibia. The original ulna mean was 291.333 and standard deviation was 240.7311. The original tibia mean was 347.457 and standard deviation was 16.23.

Mean and Standard Deviation

This shows that both devices mean and standard deviation for the ulna (a), femur (b), humerus (c), and tibia (d). Two outliers removed from the tibia and one from the ulna PaleoTech measurements. These outliers were removed due being over 50 mm from the baseline.

The inter-observer percent error rate was calculated for the volunteers using the PODv1 and the PaleoTech osteometric board (Table 5.2). Error percentage rate was calculated using the baseline value (described above) and the total average of all the volunteers' measurements for each bone element with both devices. These results show that both devices have less than a 2% error rate with each bone element. PaleoTech had an overall lower percent error rate than the PODv1. The only PaleoTech length measurement to have a higher percent error rate than the PODv1 was the tibia. These results support H2, which states that the volunteers using the POD version 1 and the Paleo-Tech Lightweight Field Osteometric Board will have measurements that exhibit an inter-observer error less than 2%, utilizing the measurement of the author as a baseline.

Error Rate for PODv1 and PaleoTech

Precent Error Rate for PODv1 and PaleoTech

| Device | Ulna | Tibia | Femur | Humerus |
|---|---|---|---|---|
| PODv1 | 0.628% | 0.8213% | 0.3773% | 0.3255% |
| PaleoTech | 0.0067% * | 1.0259% * | 0.1111% | 0.0720% |

* One outlier removed from the ulna and two from the tibia. The original tibia percent error rate was 10.421% the original ulna percent error rate was 1.0239%.

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1 AdaFruit. “Adafruit VL53L0X Time of Flight Micro-LIDAR Distance Sensor Breakout.” Adafruit Learning System. https://learn.adafruit.com/adafruit-v15310x-micro-lidar-distance-sensor-breakout/overview (Mar. 2, 2021).
2. Adams, B, and Je Byrd. (2002). “Interobserver Variation of Selected Postcranial Skeletal Measurements.” Journal of Forensic Sciences 47(6): 1193-1202.
3. Albrecht, G. (1983). “Humidity as a Source of Measurement Error in Osteometrics.” American Journal of Physical Anthropology 60(4): 517-21.
4. Anderson, Eric, and Anna Osterholtz. (2021). “The Future Is Coming: Osteometric Laser.” Presented at the Graduate Student Symposium, Mississippi State University.
5. Bräuer, Günter. (1988). Anthropologie. In R. Martin and R. Knußmann (Eds.), Handbuch Der Vergleichenden Biologie Des Menschen. 4th ed. New York: Gustav Fischer Verlag.
6. DiGangi, Elizabeth, and Megan Moore. (2012). Introduction to Skeletal Biology. In. E. A. DiGangi and M. K. Moore (Eds.), Research Methods in Human Skeletal Biology. (pp. 328). Boston, MA: Academic Press.
7 Faul, F., Erdfelder, E., Buchner, A., & Lang, A.-G. (2009). Statistical power analyses using G*Power 3.1: Tests for correlation and regression analyses. Behavior Research Methods, 41, 1149-1160
8. Geise, M. (1986). “Technical Report: The Effects of Humidity on the Abawerk Osteometric Board.” American Journal of Physical Anthropology 71(4): 485-86.
9. Harris, Edward F., and Richard N. Smith. (2009). “Accounting for Measurement Error: A Critical but Often Overlooked Process.” Archives of Oral Biology 54(Supplement 1): S107-17.
10. Hepburn, D. (1899). “A New Osteometric Board.” Journal of anatomy and physiology 34(Pt 1): 111-12.
11. Hrdlička, Aleš. (1920). Anthropometry. Philadelphia, The Wistar institute of anatomy and biology.
12. Jamison, P., and S. Zegura. (1974). “A Univariate and Multivariate Examination of Measurement Error in Anthropometry.” American Journal of Physical Anthropology 40(2): 197-203.
13. Jans, Ryan M., Adam S. Green, and Lucas J. Koerner. (2020). “Characterization of a Miniaturized IR Depth Sensor With a Programmable Region-of-Interest That Enables Hazard Mapping Applications.” IEEE Sensors Journal 20(10): 5213-20.
14. Koerner, Lucas J. (2021). “Models of Direct Time-of-Flight Sensor Precision That Enable Optimal Design and Dynamic Configuration.” IEEE Transactions on Instrumentation and Measurement 70: 1-9.
15. Langley, Natalie, L. Jantz, R. Jantz, S. Ousley, H. Maijanen, & S. McNulty. (2016). “DCP 2.0 Osteometric Data.”
16. Langley, Natalie, L. Jantz, S. McNulty, H. Maijanen, S. Ousley, & R. Jantz. (2018). “Error
17. Quantification of Osteometric Data in Forensic Anthropology.” Forensic Science International 287: 183-89.
18. Marks, Jonathan. (2012). “Why Be against Darwin? Creationism, Racism, and the Roots of
19. Anthropology.” American Journal of Physical Anthropology 149(S55): 95-104.
20. Marks, Jonathan. (2017). Is Science Racist? Malden, MA: Polity.
21. Moore, Megan. (2012). Sex Estimation and Assessment. In. E. A. DiGangi and M. K. Moore (Eds.), Research Methods in Human Skeletal Biology. (pp. 91-116). Moore. Boston, MA: Academic Press.
22. Moore, Megan, and Anna Ross. (2012). Stature Estimation. In. E. A. DiGangi and M. K. Moore (Eds.), Research Methods in Human Skeletal Biology. (pp. 151-179). Boston, MA: Academic Press.
23. Nance, Jack. (1987). Reliability, Validity, and Quantitative Methods in Archaeology. In Mark
24. Aldenderfer (Ed.), Quantitative Research in Archaeology: Progress and Prospects (pp. 245-293). Newbury Park, California: SAGE Publications, Inc.
25. Naples, Virginia L., David Breed, and Jon S. Miller. (2010). “A Skeleton Tells Its Own Story:
26. Forensic Analyses of Skeletal Elements for the Science Classroom Laboratory.” American Biology Teacher (National Association of Biology Teachers) 72(3): 162-71.
27. “Paleo-Tech Lightweight Field Osteometric Board.” Paleo-Tech Inc. https://paleo-tech.com/paleo-tech-lightweight-field-osteometric-board/(Feb. 10, 2021).
28. Perini, Talita A., G. Lamiera de Oliveira, J. dos Santos Ornellas, and F. Palha de Oliveira. (2005). “Technical Error of Measurement in Anthropometry.” Revista Brasileira de Medicina do Esporte 11(1): 81-90.
29. Schiller, Francis. (1992). Paul Broca, Founder of French Anthropology, Explorer of the Brain. New York: Oxford University Press.
30. STMicroelectronics. (2018). VL53LOX: Data Sheet. Retrieved from https://www.st.com/resource/en/datasheet/v15310x.pdf.

It will be understood that various details of the presently disclosed subject matter can be changed without departing from the scope of the subject matter disclosed herein. Furthermore, the foregoing description is for the purpose of illustration only, and not for the purpose of limitation.

We claim:

1. An osteometric measuring device comprising:
a first panel;
a second panel movably positioned with respect to the first panel; and
a light emitting device arranged and disposed in the first panel to measure distance by time-of-flight; wherein the light is emitted from the first panel, reflected off of the second panel, and received by the first and measured to calculate the distance from the first panel to the second.

2. The device of claim 1, wherein the light emitting device includes a laser transmitter and laser sensor.

3. The device of claim 2, wherein the laser transmitter and the laser sensor are positioned in the first panel.

4. The device of claim 2, wherein the laser is a red laser.

5. The device of claim 1, wherein the first panel includes a sliding feature.

6. The device of claim 5, wherein the second panel is coupled to the sliding feature.

7. The device of claim 1, wherein there is an object snugly placed between the first and second panel.

8. The device of claim 7, wherein the object is a bone.

9. The device of claim 1, wherein the second panel has a reflection target with at least 73% reflectivity.

10. The device of claim 1, wherein the second panel has a reflection target with at least 88% reflectivity.

11. The device of claim 1, wherein the measured distance between the first panel and the second panel is from about 0.03 m to about 2 m.

12. The device of claim 1, wherein the first and second panel are secured to a flat surface.

13. The device of claim 1, wherein the first and second panels are secured to the flat surface with a clamp mechanism.

14. The device of claim 1, wherein the first panel includes a display to inform the user of the distance between the first and second panel.

15. The device of claim 1, wherein the clamp mechanism includes a clamp screw, a sliding bar screw, and a sliding bar.

16. The device of claim 15, wherein at least one panel has a housing to store the clamp device.

17. The device of claim 1, wherein the first panel receives the reflected light with a sensor.

18. The device of claim 1, where the sensor includes a mirror and a light sensor below the mirror.

19. The dice of claim 1, wherein the first and second panels attached to one another for storage and transportation.

20. The device of claim 1, wherein the first and second panels engage a flexible mat for stabilization and alignment.
